(19) United States
(12) Patent Application Publication
(10) Pub. No.: US 2008/0206881 A1
(43) Pub. Date: Aug. 28, 2008

(54) DIAGNOSTIC MARKER FOR NEURODEGENERATIVE DISEASES

(75) Inventors: Ruediger HARDELAND, Rosdorf (DE); Hannelore Ehrenreich, Goettingen (DE); Klaus-Armin Nave, Goettingen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: MAX-PLANCK-GESEL. ZUR FOERDERUNG DER WISSEN. E.V, Muenchen (DE)

(21) Appl. No.: 12/038,835

(22) Filed: Feb. 28, 2008

Related U.S. Application Data
(60) Provisional application No. 60/891,970, filed on Feb. 28, 2007.

(51) Int. Cl. G01N 33/00

(52) U.S. Cl. 436/86

(57) ABSTRACT

For neurodegenerative diseases such as amyotrophic lateral sclerosis the prediction of disease progression or response to therapy based on phenotypic parameters is very difficult and inaccurate. The measurement of the serum protein carbonyl content allows a quantitative and early diagnosis of these diseases. It enables the monitoring of the disease progression as well as the individual adjustment of the therapy. Furthermore, this diagnostic marker can be used as a readout in animal model based screening methods for new therapeutic approaches and compounds.

### BACKGROUND OF THE INVENTION

1. Field of the Invention

The present invention relates to the use of serum protein carbonyl content as diagnostic marker for amyotrophic lateral sclerosis (ALS) and further neurodegenerative diseases based on a pathophysiology involving neuronal damage by oxidative stress. Serum protein carbonyl content can be used for diagnosis and as a quantitative measurement for the progression of these diseases. Furthermore, according to the invention the serum protein carbonyl content allows to judge the therapeutic outcome and to adjust the individual administration for patients and it can be used for the evaluation of new therapeutic approaches in animal models for the diseases.

### DESCRIPTION OF THE BACKGROUND

Amyotrophic lateral sclerosis (ALS) is a progressive neurodegenerative disease, affecting predominantly motoneurons in the cerebral cortex and anterior horn of the spinal cord. Dysfunction and premature death of these neurons causes spasticity, hyperreflexia, muscular atrophy, and generalized paralysis. Respiratory failure is the main cause of death within 2-5 yr after diagnosis. Most ALS cases occur sporadic. Among the 5-10% familial forms, about 20% are associated with mutations in the gene for superoxide dismutase (SOD1) (Rowland New Engl J Med 2001: 344: 1688-1700).

To date, there is no causal or convincing symptomatic treatment for ALS. The only compound with borderline efficacy in ALS patients is RILUZOLE® (2-amino-6-(trifluoromethoxxy)benzothiazole), an antiexcitotoxic drug that marginally prolonged survival in clinical trials (Bensimon New Engl J Med 1994: 330: 585-591; Lacomblez Lancet 1996: 347: 1425-1431), but lacked benefit with respect to other important outcome measures. Owing to its side effects (including asthenia, nausea, anorexia, diarrhea, headache, and increase in liver transaminases), RILUZOLE® is frequently discontinued. It also enhances catabolism in this already wasting disease (Bensimon Expert Opin Drug Saf 2004: 3: 525-534). Thus, when screening for new drugs in ALS, it is important to search for well-tolerated compounds that can be rapidly transferred to a clinical setting.

All mechanisms believed to explain neuronal cell death or dysfunction in ALS involve oxidative stress mediated by reactive nitrogen/oxygen species, either as primary insult or as part of the final common pathway of disease (Bruijn Annu Rev Neurosci 2004: 27: 723-749; Reiter Prog Neurobiol 1998: 56: 359-384; Tan Curr Top Med Chem 2002: 2: 181-197). A recent 10-yr prospective study with over 900,000 individuals showed a decreased risk of developing ALS that was associated with the regular intake of vitamin E (Ascherio Ann Neurol 2005: 57: 104-110). Nevertheless, none of the antioxidative compounds tested, including vitamin E, proved to be effective in the clinical setting [Rowland New Engl J Med 2001: 344: 1688-1700; Desnuelle ALS riluzole-tocopherol Study Group. Amyotroph Lateral Scler Other Motor Neuron Disord 2001:2:9-18; Graf J Neural Transm 2005: 112: 649-660).

Accordingly for ALS, there remains a prominent need for early intervention and better compounds against oxidative stress.

Melatonin is distinct from classical antioxidants. First, it acts on a unique broad spectrum of free radical targets by direct scavenging (Hardeland Trends Comp Biochem Physiol 1996: 2: 25-45; Reiter Prog Neurobiol 1998: 56: 359-384; Tan Curr Top Med Chem 2002: 2: 181-197). Second, its antioxidative profile extends to the activation of other antioxidative systems, such as glutathione peroxidase. Third, melatonin is amphiphilic, and in contrast to standard antioxidants, enters both lipophilic and hydrophilic cellular environments. Fourth, melatonin attenuates radical formation directly, and indirectly through its kynuramine metabolites, by antiexcitotoxic, antiinflammatory and mitochondrial mechanisms (Hardeland Endocrine 2005: 27: 119-130; Hardeland Nutr Metab (Lond) 2005: 2: article no 22).

The prolongation of survival of an ALS mouse model due to a high-dose oral melatonin treatment has been described (U.S. Pub. No. 2004/0192745). Therefore, the daily administration of 300-600 mg is suggested for the treatment of human ALS patients.

Further to ALS oxidative stress mediated by reactive oxygen species have been implicated also in the pathophysiology of several other neurological diseases, such as Alzheimer's disease, Parkinson's disease, Huntington's disease, and Friedreich's ataxia (Piemonte Eur J Clin Invest 2001: 31: 1007-1011; Beal Free Radic Biol Med. 2002: 32: 797-803).

Alzheimer's disease (AD) is the most common neurodegenerative disease. Clinically, it leads to progressive memory loss and dementia. The neuropathological hallmarks are senile plaques containing β-amyloid and neurofibrillary tangles, which occur in pyramidal neurons of the cerebral cortex and hippocampus. In AD there is a large body of evidence implicating oxidative damage. Furthermore, administration of vitamin E leads to slowing of disease progression. There is also epidemiologic evidence that pations taking antioxidant vitamins and anti-inflammatory compounds have a lower incidence of AD. Several biochemical studies showed increased concentrations of protein carbonyls in AD patients in both hippocampus and the inferior parietal lobule, but unchanged concentrations in the cerebellum, which is consistent with the regional pattern of histopathology in AD (Beal Free Radic Biol Med. 2002: 32: 797-803; Hensley J Neurochem. 1995: 65: 2146-56).

Parkinson's disease (PD) is the second most common neurodegenerative disease. It causes a progressive movement disorder. There is a loss of substantia nigra dopaminergic neurons. The histopathologic hallmark is eosinophilic cytoplasmatic inclusions in the substantia nigra neurons known as Lewy bodies. In PD increases in protein carbonyls were found in all brain regions examined including the substantia nigra, basal ganglia, globus pallidus, substantia innominata, frontal cortex, and cerebellum. An explanation would be a widely expressed genetic defect in the brain leading to oxidative damage. Furthermore, there is substantial evidence implicating peroxynitrite-induced protein damage in PD and in animal models of PD. Increased 3-nitrotyrosine which is thought to be a relative specific marker of oxidative damage mediated by peroxynitrite, was shown in Lewy bodies and in amorphous deposits in intact and degenerating neurons in PD substantia nigra (Beal Free Radic Biol Med. 2002: 32: 797-803).

Huntington's disease (HD) is an autosomal dominant inherited neurodegenerative disease in which there is both a movement disorder and dementia. Neuropathologically, the damage predominates in the basal ganglia. In HD increased oxidative damage to DNA has been found in both HD postmortem tissue and a transgenic mouse model. Increased 3-nitrotyrosine immunostaining has also been reported in a transgenic mouse model of HD (Beal Free Radic Biol Med. 2002: 32: 797-803).

In recent years, oxidative stress has been proposed as a pathogenic factor for Friedreich's ataxia (FRDA), an autosomal, recessive, neurodegenerative disease caused by a deficiency of frataxin, a highly conserved mitochondrial protein. Most patients (95%) are homozygous for the hyperexpansion of a GAA repeat sequence in the first intron of the frataxin gene, a few are heterozygous for a GAA expansion and a point mutation.

Patients frequently develop cardiomyopathy, diabetes mellitus and skeletal abnormalities (Piemonte Eur J Clin Invest 2001: 31: 1007-1011).

This suggests that new therapeutic approaches employing suitable antioxidative agents such as melatonin may be promising for the treatment of ALS, other related motor neuron diseases, and other neurodegenerative diseases based on a pathophysiology involving neuronal damage by oxidative stress (Srinivasan Neurotox Res 2005: 7: 293-318; Srinivasan Behav Brain Funct 2006: 2: article no 15).

Therefore, accessible diagnostic markers need to be defined to enable an early intervention, a better adjustment of the individual treatment protocol, a better evaluation of the therapeutic outcome, and a better prediction of the efficacy of new compounds against oxidative stress.

An increased protein carbonyl content in the involved brain tissue has been demonstrated as an evidence for the oxidative damage associated with ALS (Ferrante J Neurochem 1997: 69: 2064-2074) and other neurodegenerative diseases (Beal Free Radic Biol Med. 2002: 32: 797-803).

However, serum protein carbonyl measurement has not been described for its use as accessible diagnostic marker for ALS or other related neurodegenerative diseases

### SUMMARY OF THE INVENTION

Accordingly, it is an object of the present invention to provide a new diagnostic marker for ALS or other related neurodegenerative diseases comprising the measurement of the serum protein carbonyl content.

The serum protein carbonyl content as diagnostic marker for ALS or other related neurodegenerative diseases can be easily monitored from the peripheral blood samples from the patients.

According to the present invention, the monitoring of the serum protein carbonyl content of patients is suitable for the diagnosis of ALS or other related neurodegenerative diseases, for the evaluation of the therapeutic outcome, and for the adjustment of the individual treatment protocol.

Further according to the present invention, the monitoring of the serum protein carbonyl content of animal models of ALS or other related neurodegenerative diseases is suitable for the discovery and for the evaluation of new therapeutic approaches and potential pharmaceutical compounds.

ALS related neurodegenerative diseases according to the invention are neurodegenerative diseases which are based on a pathophysiology involving neuronal damage by oxidative stress. Such diseases are for example but not limited to Alzheimer's disease, Parkinson's disease, Huntington's disease, and Friedreich's ataxia

### BRIEF DESCRIPTION OF THE DRAWINGS

FIG. 1 is a depiction of the serum protein carbonyl content depending on the treatment of amyotrophic lateral sclerosis (ALS) patients with melatonin. Elevated protein carbonyl content in the serum of untreated ALS patients (n=19) decrease to levels of matched healthy controls (n=10) upon melatonin treatment (mean treatment time: 4.68±0.22 months). Mean±S.E.M. presented.

### DETAILED DESCRIPTION OF THE INVENTION

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Oxidative stress mediated by reactive nitrogen/oxygen species has been implicated in the pathophysiology of ALS and other related neurodegenerative diseases such as AD, PD, HD, and FRDA. ALS autopsy material contains increased levels of protein carbonyls in the brain tissue, representing protein modifications caused by the oxidative stress.

However, protein carbonyl content obtained from serum has not been accessed for its properties as diagnostic marker for neurodegenerative diseases such as ALS. It seems unlikely that carbonyl modified proteins from the oxidatively damaged brain tissue penetrate the blood brain barrier to reach the blood circulation.

Unexpectedly, the inventors have found that circulating serum protein carbonyls provide a diagnostic surrogate marker for oxidative stress and were elevated in ALS patients. Moreover, the serum protein carbonyls were normalized to control values by melatonin treatment, which has been shown to exhibit efficacy in studies with an ALS mouse model (U.S. Pub. No. 2004/0192745). Probably, the elevated peripheral protein carbonyls are not derived from the central nervous system, but they appear to reflect systemic oxidative stress in ALS patients. Protein carbonyl content of blood serum and other body fluids can be determined according to the 2,4-dinitrophenylhydrazine method described by Levine et al. (Levine Methods Enzymol 1990: 186: 464-478). The protein content can be measured according to Lowry et al. (Lowry J Biol Chem 1951: 193: 265-275) to enable a normalization of the protein carbonyl level. Other suitable methods and reagents known in the field to measure protein content and/or the content of protein carbonyls can also be used.

The elevation of protein carbonyls can be monitored in peripheral blood samples from ALS patients, and can be used as a biochemical readout for a therapeutic effect. Small but significantly elevated protein carbonyl concentrations are detectable in serum of ALS patients compared with the serum of matched healthy controls. Significantly, by follow-up of the same ALS patients after the melatonin treatment, the protein carbonyl levels are fully reverted to control levels. Remarkably, treatment of ALS patients with high-dose vitamin E, another antioxidant, does not change the level of serum protein carbonyls. In fact, a comparison of the clinical outcome of ALS patients with or without continuous high-dose vitamin E, did not reveal any beneficial effect of vitamin E (Rowland New Engl J Med 2001: 344: 1688-1700; Desnuelle ALS riluzole-tocopherol Study Group. Amyotroph Lateral Scler Other Motor Neuron Disord 2001: 2: 9-18; Graf J Neural Transm 2005: 112: 649-660).

The increased level of serum protein carbonyl is indicative for ALS, moreover, it is suitable to predict the response to a therapy. Although melatonin as well as vitamin E are antioxidative compounds, only the promising melatonin treatment normalizes the serum protein carbonyl level to control level. Vitamin E which is meanwhile known to be ineffective in ALS treatment does not decrease the serum protein carbonyl level of the ALS patients. The serum protein carbonyl content does not simply reflect the systemic effect of antioxidative compounds on reactive nitrogen/oxygen species, it rather reflects the therapeutic benefit of certain antioxidative compounds.

Therefore, the present invention indicates that the serum protein carbonyl level is a valuable diagnostic marker for neurodegenerative diseases which have a pathophysiology involving neuronal damage by oxidative stress.

By “diagnostic marker” is meant an easily accessible biological substance which can be preferably obtained e.g. from blood or other body fluids. The diagnostic marker exhibits a detectable qualitative or quantitative change which is indicative for a disease, predictive for a response to therapy, and/or predictive for disease progression. Furthermore, a diagnostic marker can be utilized as a surrogate marker to predict the efficacy of a potential new treatment in an animal model for the disease. Only an easily accessible biological substance can be reasonably regarded as a diagnostic marker.

In one embodiment of the present invention, the serum protein carbonyl determination is utilized to diagnose neurodegenerative diseases with a pathophysiology involving neuronal damage by oxidative stress, such as e.g. ALS, AD, HD, PD, or FRDA. Preferably, the serum protein carbonyl determination is utilized to diagnose ALS. The protein carbonyl determination enables an early diagnosis of the neurodegenerative disease before the onset of clinical symptoms. This, in turn, allows for an early therapy onset which seems to be crucial for the therapeutic efficacy (Ludolph J Neurol 2000: 247:VI/13-VI/18). In one aspect of this embodiment, an increased level of serum protein carbonyls in a test subject relative to a negative standard, e.g., a subject known not to be afflicted by the disease is diagnostic that the test subject has or will have a neurodegenerative disease with a pathophysiology involving neuronal damage by oxidative stress as discussed herein. The increased level can be any appreciable increase in relative serum levels of the protein carbonyls, including, e.g., at least a 5% increase, 10, 20, 30, 50, 75 and at least 100% increase or more.

In another embodiment of the present invention, the serum protein carbonyl determination is utilized to monitor the progress of a therapy of patients with neurodegenerative diseases with a pathophysiology involving neuronal damage by oxidative stress, such as e.g. ALS, AD, HD, PD, or FRDA. Preferably, the serum protein carbonyl determination is utilized to monitor the therapy of ALS patients. The large variety of clinical symptoms during the progression of these diseases inhibits a practical evaluation of the individual response to the therapy. Furthermore, the effects of the therapy will occur very delayed. This makes it nearly impossible to adjust the therapy (e.g. choice of therapeutic compound, administration method, and dosage) individually to the patient. In contrast to this, the serum protein carbonyl determination enables a quantitative and early evaluation of the individual response to the therapy and consequently the individual adjustment of the therapy. In one aspect of this embodiment, a decreased level of serum protein carbonyls in a test subject relative to a the same or similar subject prior to the onset of therapy and/or who has not received therapy is indicative that the therapy is at least partially effective at treating the disease as discussed herein. In another embodiment, levels that are approximately the same relative to a the same or similar subject prior to the onset of therapy and/or who has not received therapy is indicative that the therapy is not at least partially effective at treating the disease as discussed herein. The increased or decreased levels can be any appreciable increase in relative serum levels of the protein carbonyls, including, e.g., at least a 5% increase, 10, 20, 30, 50, 75 and at least 100% increase/decrease or more.

In another embodiment of the present invention, the serum protein carbonyl determination is utilized to predict the efficacy of potential new treatments in animal models for the neurodegenerative diseases with a pathophysiology involving neuronal damage by oxidative stress, such as e.g. ALS, AD, HD, PD, or FRDA. Preferably, the serum protein carbonyl determination is utilized to evaluate new approaches of ALS treatment. A quantitative evaluation based on the clinical symptoms of the test animals is difficult. Furthermore, the effects of the treatment occur delayed which make such analyses time-consuming. In contrast to this, the determination of serum protein carbonyl from the test animals enables a quantitative and early evaluation of the efficacy of the new treatment. In one aspect of this embodiment, a decreased level of serum protein carbonyls in a test animal relative to a the same or similar animal prior to the onset of therapy testing and/or who has not received therapy is indicative that the therapy is at least partially effective at treating the disease as discussed herein. In another embodiment, levels that are approximately the same relative to a the same or similar animal tested prior to the onset of therapy and/or who has not received therapy is indicative that the therapy is not at least partially effective at treating the disease as discussed herein. The increased or decreased levels can be any appreciable increase in relative serum levels of the protein carbonyls, including, e.g., at least a 5% increase, 10, 20, 30, 50, 75 and at least 100% increase/decrease or more.

Suitable animal models for neurodegenerative diseases are described in literature. Non-limiting examples of such animal models are SOD1G93A-transgenic mouse for ALS (Almer J Neurochem 1999: 72: 2415-2425), 6-hydroxydopamine treated rat for PD (Zigmond Life Sci 1984: 35: 5-18), MPTP treated rat for PD (Allen Brain 1986: 109(Pt1): 143-157), APP-transgenic mice for AD (Janus Physiol Behav 2001: 73: 873-886), exon-1-huntingtin-transgenic mice for HD (Sathasivam Philos Trans R Soc Lond B Biol Sci. 1999: 354: 963-969), and FRDA-transgenic mice for FRDA (Al-Mahdawi Genomics 2006: 88: 580-590).

### EXAMPLES

### Example 1

### Protein Carbonyl Determination

Protein carbonyl measurements were performed in the serum of 19 consecutively admitted ALS patients (61.7±1.9 yr, 12 males, seven females) before and after ≧24 months of melatonin treatment (300 mg melatonin applied daily as suppositories at bedtime) and of ten healthy controls (61.2±2.0 years, five males, five females). Protein carbonyls were determined by a variant of the 2,4-dinitrophenylhydrazine method (Levine Methods Enzymol 1990: 186: 464-478). Serum was diluted 1:10 with ice-cold 5-mM pf potassium phosphate buffer, pH 7.5, containing leupeptin (0.5 μg/mL), aprotinin (0.5 μg/mL), and pepstatin A (0.7 μg/mL); 300 μL was used for a single determination. Deviations from the original procedure concerned elimination of the chromogen: protein pellets were washed three times with 1 mL of ethanol/ ethylacetate (1:1); particular attention was given to thorough resuspension (vortexing) and complete removal of the supernatant using Pasteur pipettes, and thereafter, stripes of blotting paper. Protein was measured according to Lowry et al. (Lowry J Biol Chem 1951: 193: 265-275).

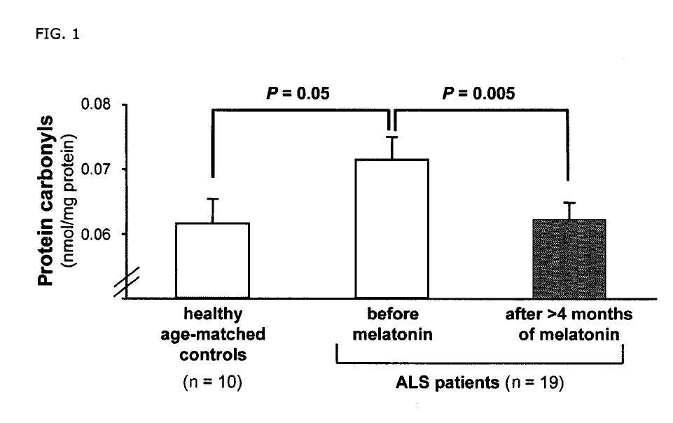

Small but significantly elevated protein carbonyl concentrations in serum of ALS patients compared with the serum of matched healthy controls have been measured. Significantly, by follow-up of the same ALS patients more than 4 months after the start of daily melatonin treatment, protein carbonyl levels have been found fully reverted to control levels (FIG. 1). Remarkably, most of these patients (n=12) had already been on high-dose vitamin E, another antioxidant, for 10.4±2.5 months before starting the melatonin treatment. In fact, a comparison of the clinical outcome (ALSFRS after 1 yr of melatonin treatment) of ALS patients with or without continuous high-dose vitamin E, did not reveal any beneficial effect of vitamin E.

1. A method of predicting response to therapy or predicting disease progression in neurodegenerative diseases, the method comprising:
obtaining a serum sample from a subject; and
determining the protein carbonyl content of said serum sample.

2. The method of claim 1, wherein the protein carbonyl content is compared to that one of healthy controls.

3. The method of claim 1, wherein the neurodegenerative disease has a pathophysiology involving neuronal damage by oxidative stress.

4. The method of claim 1, wherein the neurodegenerative disease is amyotrophic lateral sclerosis, Alzheimer's disease, Parkinson's disease, Huntington's disease, or Friedreich's ataxia.

5. The method of claim 1, wherein the neurodegenerative disease is amyotrophic lateral sclerosis.

6. The method of claim 1, wherein the subject is a human.

7. The method of claim 1, wherein the subject is an animal model for said neurodegenerative disease.

8. The method of claim 7, wherein the protein carbonyl content is determined from said animal model after the administration of a potential therapeutic preparation as a readout for the efficacy of said preparation.

9. The method of claim 1, wherein the serum sample is obtained from peripheral blood.

10. A method of diagnosing neurodegenerative diseases, the method comprising:
obtaining a serum sample from a subject;
determining the protein carbonyl content of said serum sample; and
comparing said protein carbonyl content to that one of healthy controls.
